(19) United States
(12) Patent Application Publication
(10) Pub. No.: US 2025/0362315 A1
(43) Pub. Date: Nov. 27, 2025

(54) QUANTIFICATION OF LIPOPROTEIN SUBFRACTION BY ION MOBILITY

(71) Applicants: Michael J. MCPHAUL, Capistrano Beach, CA (US); Charles M. ROWLAND, San Leandro, CA (US); Quest Diagnostics Investments LLC, Secaucus, NJ (US)

(72) Inventors: Michael J. McPhaul, Capistrano Beach, CA (US); Charles M. Rowland, San Leandro, CA (US)

(21) Appl. No.: 18/872,501

(22) PCT Filed: Jun. 8, 2023
(86) PCT No.: PCT/US2023/024856

§ 371 Date: Dec. 6, 2024

Related U.S. Application Data
(60) Provisional application No. 63/350,789, filed on Jun. 9, 2022.

(51) Int. Cl. G01N 33/92

(52) U.S. Cl. CPC G01N 33/92; G01N 2800/042

(57) ABSTRACT

Methods are provided for quantification of lipoprotein subfraction by ion mobility. Also provided herein are methods for diagnosing or prognosing insulin resistance in a patient in need thereof (e.g., diabetic and/or pre-diabetic patients), the method comprising measuring lipoprotein subfraction levels in a patient sample by ion mobility.

### CROSS REFERENCE TO RELATED PATENT APPLICATIONS

This application claims benefit of U.S. Provisional Application No. 63/350,789, filed Jun. 9, 2022, which is incorporated by reference herein in its entirety.

### FIELD OF THE INVENTION

The invention relates to the identification and quantitation of lipoprotein subfractions by ion mobility and determining the risk of insulin resistance.

### BACKGROUND OF THE INVENTION

Insulin resistance (IR) is associated with lipid and lipoprotein abnormalities including high triglycerides (TG) and low high-density lipoprotein cholesterol (HDL-C) that contribute to increased risk of atherosclerotic cardiovascular disease. Direct measurement of IR is labor-intensive and cannot be performed in a clinical setting. Thus, an effective method of determining the risk of insulin resistance is needed.

### SUMMARY OF THE INVENTION

In one aspect, provided herein are methods for diagnosing or prognosing insulin resistance in a patient in need thereof (e.g., diabetic and/or pre-diabetic patients), the method comprising measuring lipoprotein subfraction levels in a patient sample by ion mobility. Also provided are methods for determining the amount of lipoprotein subfraction in a sample, the method comprising determining the amount of the lipoprotein subfraction in the sample by ion mobility.

In certain embodiments, the methods provided herein comprise lipoprotein subfraction in a sample by ion mobility.

In some embodiments, an insulin resistance score (RS) and/or probability of developing insulin resistance, P(IR), is provided herein based on the levels of lipoprotein subfraction measured by methods provided herein.

In some embodiments, an insulin resistance score (RS) and/or probability of developing insulin resistance, P(IR), is provided herein based on the levels of lipoprotein subfraction measured by methods provided herein and triglyceride (TG) and high density lipoprotein cholesterol (HDL-C) levels.

In some embodiments, an insulin resistance score (RS) and/or probability of developing insulin resistance, P(IR), is provided herein based on the levels of lipoprotein subfraction measured by methods provided herein and triglyceride (TG) and high-density lipoprotein cholesterol (HDL-C) levels, in combination with sex, race, ethnicity and body mass index (BMI) measured by standard methods.

In certain embodiments, insulin resistance is diagnosed by the levels of lipoprotein subfraction measured by methods provided herein. In some embodiments, insulin resistance is diagnosed by the levels of lipoprotein subfraction measured by methods provided herein and triglyceride (TG) and high density lipoprotein cholesterol (HDL-C) levels. In some embodiments, insulin resistance is diagnosed by the levels of lipoprotein subfraction measured by methods provided herein and triglyceride (TG) and high density lipoprotein cholesterol (HDL-C) levels, in combination with sex, race, ethnicity and body mass index (BMI) measured by standard methods.

As used herein, unless otherwise stated, the singular forms “a,” “an,” and “the” include plural reference. Thus, for example, a reference to “a protein” includes a plurality of protein molecules.

The term “purification” or “purifying” refers to a procedure that enriches the amount of one or more analytes of interest relative to other components in the sample that may interfere with detection of the analyte of interest. Although not required, “purification” may completely remove all interfering components, or even all material other than the analyte of interest. Purification of the sample by various means may allow relative reduction of one or more interfering substances, e.g., one or more substances that may or may not interfere with the detection of selected parent or daughter ions by mass spectrometry. Relative reduction as this term is used does not require that any substance, present with the analyte of interest in the material to be purified, is entirely removed by purification.

The term “sample” refers to any sample that may contain an analyte of interest. As used herein, the term “body fluid” means any fluid that can be isolated from the body of an individual. For example, “body fluid” may include blood, plasma, serum, bile, saliva, urine, tears, perspiration, and the like. In preferred embodiments, the sample comprises a body fluid sample; preferably plasma or serum.

An “amount” of an analyte in a body fluid sample refers generally to an absolute value reflecting the mass of the analyte detectable in volume of sample. However, an amount also contemplates a relative amount in comparison to another analyte amount. For example, an amount of an analyte in a sample can be an amount which is greater than a control or normal level of the analyte normally present in the sample.

The term “about” as used herein in reference to quantitative measurements not including the measurement of the mass of an ion, refers to the indicated value plus or minus 10%.

The summary of the invention described above is non-limiting and other features and advantages of the invention will be apparent from the following detailed description of the invention, and from the claims.

### BRIEF DESCRIPTION OF THE DRAWINGS

FIG. 1A shows the relationship among insulin resistance, BMI, and LS-IM score SSPG concentration by tertiles (T) of LS-IM Score on x-axis (T1: <−5.7, T2: >=−5.7 to <8.04, T3: >=8.04) and BMI in panels (T1: <27.7 kg/m², T2: >27.7 to <=32.1 kg/m², T3: >32.1 kg/m²). FIG. 1B shows the relationship among insulin resistance, BMI, and TG/HDL-C SSPG concentration by tertiles (T) of LS-IM Score on x-axis (T1: <−5.7, T2: >=−5.7 to <8.04, T3: >=8.04) and BMI in panels (T1: <27.7 kg/m², T2: >27.7 to <=32.1 kg/m², T3: >32.1 kg/m²). FIG. 1C shows the relationship among insulin resistance, BMI, and LS-IM score+TG/HDL-C SSPG concentration by tertiles (T) of LS-IM Score on x-axis (T1: <−5.7, T2: >=−5.7 to <8.04, T3: >=8.04) and BMI in panels (T1: <27.7 kg/m², T2: >27.7 to <=32.1 kg/m², T3: >32.1 kg/m²). The number of subjects are printed below each box plot. The SSPG concentration is the direct measure of insulin resistance where a higher SSPG concentration indicates greater degree of insulin resistance than a lower SSPG concentration. LS-IM Score=−8.8*VLDL Medium−19*IDL Small+11*LDL Large a+10.2*LDL Medium+14.8*LDL very small b−16.8*LDL very small c+8.6*LDL very small d+7.9*HDL small with all LS-IM values in standard deviation units and standard deviations of 15.8, 50.3, 92.6, 8.1, 62.6, 34.6, 19.5 and 3267 nmol/L, respectively. LS-IM Score+TG/HDL-C=−8.8*VLDL Medium−19*IDL Small+11*LDL Large a+10.2*LDL Medium+14.8*LDL very small b−16.8*LDL very small c+8.6*LDL very small d+7.9*HDL small+17.1*TG/HDL-C with all LS-IM values in standard deviation units and standard deviations of 15.8, 50.3, 92.6, 8.1, 62.6, 34.6, 19.5, 3267 nmol/L, respectively and TG/HDL-C in standard deviation units with standard deviation of 17.1. SSPG: Steady-state plasma glucose. LS-IM: Ion mobility based subfractionation. HDL: High-density lipoprotein. LDL: Low-density lipoprotein. IDL: Intermediate-density lipoprotein. VLDL: Very low-density lipoprotein. TG/HDL-C: Ratio of triglycerides to high density lipoprotein cholesterol concentration.

FIG. 2A shows receiver operating characteristic curves to predict SSPG concentration in top tertile (>196 mg/dL): TG/HDL-C, LS-IM Score and TG/HDL-C+LS-IM Score. FIG. 2B shows receiver operating characteristic curves to predict SSPG concentration in top tertile (>196 mg/dL): BMI, BMI+TG/HDL-C and BMI+TG/HDL-C+LS-IM Score. FIG. 2C shows receiver operating characteristic curves to predict SSPG concentration in top tertile (>196 mg/dL): BMI+TG/HDL-C+Sex+Race+Ethnicity and BMI+TG/HDL-C+Sex+Race+Ethnicity+LS-IM Score. LS-IM Score=−8.8*VLDL Medium−19*IDL Small+11*LDL Large a+10.2*LDL Medium+14.8*LDL very small b−16.8*LDL very small c+8.6*LDL very small d+7.9*HDL small. TG/HDL-C+LS-IM Score=17.1*TG/HDL-C+LS-IM Score. BMI+TG/HDL-C=33.5*BMI+17.1*TG/HDL-C. BMI+TG/HDL-C=33.5*BMI+17.1*TG/HDL-C+LS-IM Score. BMI+TG/HDL+Sex+Race+Ethnicity=33.5*BMI+17.1*TG/HDL-C−12*Male+15.7*Hispanic+5.3*Native American+21.5*East Asian−6*Black+21*South Asian. BMI+TG/HDL+Sex+Race+Ethnicity+LS-IM Score=33.5*BMI+17.1*TG/HDL-C−12*Male+15.7*Hispanic+5.3*Native American+21.5*East Asian−6*Black+21*South Asian+LS-IM Score. SSPG: Steady-state plasma glucose. LS-IM: Ion mobility based lipoprotein subfractionation. HDL: High-density lipoprotein. LDL: Low-density lipoprotein. IDL: Intermediate-density lipoprotein. VLDL: Very low-density lipoprotein. TG/HDL-C: Ratio of triglycerides to high density lipoprotein cholesterol concentration.

FIG. 3 shows a flow diagram of study inclusion criteria.

### DETAILED DESCRIPTION OF THE INVENTION

Disclosed herein are methods for diagnosing or prognosing insulin resistance in a patient in need thereof (e.g., diabetic and/or pre-diabetic patients). For example, without being bound by theory, individuals in the top tertile of steady-state plasma glucose (SSPG) concentration of a given population can be defined as being insulin resistant.

In some embodiments, an individual is insulin resistant if SSPG concentration is ≥190 mg/dL. In some embodiments, an individual is insulin resistant if SSPG concentration is ≥195 mg/dL, such as ≥196 mg/dL. In some embodiments, an individual is insulin resistant if SSPG concentration is ≥198 mg/dL In some embodiments, an individual is insulin resistant if SSPG concentration is ≥200 mg/dL. In some embodiments, an individual is insulin resistant if SSPG concentration is ≥205 mg/dL.

Suitable test samples for use in methods of the present invention include any test sample that may contain the analyte of interest. In some preferred embodiments, a sample is a biological sample; that is, a sample obtained from any biological source, such as an animal, a cell culture, an organ culture, etc. In certain preferred embodiments, samples are obtained from a mammalian animal, such as a dog, cat, horse, etc. Particularly preferred mammalian animals are primates, most preferably male or female humans. Preferred samples comprise bodily fluids such as blood, plasma, serum, saliva, cerebrospinal fluid, or tissue samples; preferably plasma and serum. Such samples may be obtained, for example, from a patient; that is, a living person, male or female, presenting oneself in a clinical setting for diagnosis, prognosis, or treatment of a disease or condition.

In certain embodiments, the methods provided herein comprise lipoprotein subfraction in a sample by ion mobility. For example, the levels of lipoprotein subfraction (lipoprotein analysis) may be measured according to U.S. Patent Application Publication No. 2008/0305549 and Mora S., et al. Circulation. 2015 Dec. 8; 132 (23):2220-9, each of which is incorporated herein by reference . . . .

In some embodiments, lipoprotein subfraction comprises determining the amount of one or more or all of VLDL (very low-density lipoprotein), IDL (intermediate-density lipoprotein), LDL (low-density lipoprotein), and HDL (high-density lipoprotein). In some embodiments, lipoprotein subfraction comprises determining the amount of one or more or all of VLDL (very low-density lipoprotein) Medium, IDL (intermediate-density lipoprotein) Small, LDL (low-density lipoprotein) Large a, LDL (low-density lipoprotein) Medium, LDL (low-density lipoprotein) Very Small b, LDL (low-density lipoprotein) Very Small c, LDL (low-density lipoprotein) Very Small d, and HDL (high-density lipoprotein) Small.

In some embodiments, an insulin resistance score (RS) and/or probability of developing insulin resistance, P(IR), is provided herein based on the levels of lipoprotein subfraction measured by methods provided herein. In some embodiments, an insulin resistance score (RS) can be determined from Equation A1.

$$R S = LS - IM Score = - 8.8 * VLDL Medium - 19 * IDL Small + 11 * LDL Large a + 10.2 * LDL Medium + 14.8 * LDL very small b - 16.8 * LDL very small c + 8. 6 * LDL very small d + 7.9 * HDL small \qquad Equation A1$$

In some embodiments, the methods provided herein comprise measuring triglyceride (TG) levels and/or high density lipoprotein cholesterol (HDL-C) levels. Accordingly, in some embodiments, an insulin resistance score (RS) and/or probability of developing insulin resistance, P(IR), is provided herein based on the levels of lipoprotein subfraction measured by methods provided herein and triglyceride (TG) and high density lipoprotein cholesterol (HDL-C) levels. In some embodiments, an insulin resistance score (RS) can be determined from Equation A2. The levels of triglyceride (TG) and high density lipoprotein cholesterol (HDL-C) can be measured according to methods known in the art.

$$RS = LS - IM Score + TG / HDL - C = LS - IM Score + 17.1 * TG / HDL - C = - 8.8 * VLDL Medium - 19 * IDL Small + 11 * LDL Large a + 10.2 * LDL Medium + 14.8 * LDL very small b - 16.8 * LDL very small c + 8.6 * LDL very small d + 7.9 * HDL small + 17.1 * TG / HDL - C \qquad Equation A2$$

In some embodiments, the methods provided herein comprise measuring body mass index (BMI) or measuring body mass index (BMI) in combination with sex, race, and ethnicity. Accordingly, in some embodiments, an insulin resistance score (RS) and/or probability of developing insulin resistance, P(IR), is provided herein based on the levels of lipoprotein subfraction measured by methods provided herein and triglyceride (TG) and high-density lipoprotein cholesterol (HDL-C) levels, in combination with sex, race, ethnicity and body mass index (BMI) measured by standard methods. In some embodiments, an insulin resistance score (RS) can be determined from Equation A3. The levels of triglyceride (TG) and high density lipoprotein cholesterol (HDL-C) can be measured according to methods known in the art.

$$RS = LS - IM Score + B MI + TG / HDL - C + Sex + Race + Ethnicity = LS - IM Score + 33.5 * BMI + 17.1 * TG / HDL - C - 12 * Male + 15.7 * Hispanic + 5.3 * Native American + 21.5 * East Asian - 6 * Black + 21 * South Asian = - 8.8 * VLDL Medium - 19 * IDL Small + 11 * LDL Large a + 10.2 * LDL Medium + 14.8 * LDL very small b - 16.8 * LDL very small c + 8.6 * LDL very small d + 7.9 * HDL small + 33.5 * BMI + 17.1 * TG / HDL - C - 12 * Male + 15.7 * Hispanic + 5.3 * Native American + 21.5 * East Asian - 6 * Black + 21 * South Asian \qquad Equation A3$$

Note: in Equation A3, variable Male=1 if subject is male, variable Male=0 if subject is female; variable Hispanic=1 if subject is Hispanic, variable Hispanic=0 otherwise; variable Native American=1 if subject is Native American, variable Native American=0 otherwise; variable East Asian=1 if subject is East Asian, variable East Asian=0 otherwise; variable Black South=1 if subject is Black South, variable Black South=0 otherwise; variable Asian=1 if subject is Asian, variable Asian=0 otherwise.

In certain embodiments, insulin resistance is diagnosed by the levels of lipoprotein subfraction measured by methods provided herein. In some embodiments, insulin resistance is diagnosed by the levels of lipoprotein subfraction measured by methods provided herein and triglyceride (TG) and high density lipoprotein C (HDL-C) levels. In some embodiments, insulin resistance is diagnosed by the levels of lipoprotein subfraction measured by methods provided herein and triglyceride (TG) and high density lipoprotein C (HDL-C) levels, in combination with sex, race, ethnicity and body mass index (BMI) measured by standard methods.

In certain embodiments, the method described herein provides an insulin resistance score (e.g., according to Equation A1, Equation A2, or Equation A3).

In certain embodiments, the method described herein provides a probability of developing insulin resistance.

In certain embodiments, the biological samples provided herein comprise a plasma or serum sample.

### EXAMPLES

### Example 1: Quantification of Lipoprotein Subfraction by Ion Mobility

We assessed the usefulness of fasting lipoprotein subfractions (LS) to identify individuals with IR.

Lipid panel, LS by ion mobility (LS-IM), and IR by steady-state plasma glucose (SSPG) concentration were assessed in 526 adult volunteers without diabetes. IR was defined as being in the highest tertile of SSPG concentration. LS-IM score was calculated by linear combination of regression coefficients from a stepwise regression analysis with SSPG concentration as the dependent variable. Scores were also calculated for LS-IM score+TG/HDL-C and for a model with sex, race, ethnicity, BMI, TG/HDL-C and the LS-IM score. IR prediction was evaluated by area under the receiver operating characteristic curve (AUC) and positive predictive value (PPV) considering the highest 5% of scores as positive test.

Prediction of IR was similar by LS-IM score and TG/HDL-C(AUC=0.68; PPV=0.59 and AUC=0.70; PPV=0.59, respectively), and prediction was improved when LS-IM was combined with TG/HDL-C(AUC=0.73; PPV=0.70), TG/HDL-C and BMI (AUC=0.82; PPV=0.81) and with TG/HDL-C, BMI, sex, race and ethnicity (AUC=0.84; PPV=0.89).

For identifying individuals with IR, LS-IM score and TG/HDL-C are comparable and their combination with sex, race, ethnicity and BMI further improves IR prediction by TG/HDL-C alone. Among patients who have undergone IM testing, the LS-IM score may assist prioritization of subjects for further evaluation and interventions to reduce IR.

Insulin resistance (IR) increases risk of type 2 diabetes (T2D) and atherosclerotic cardiovascular disease (ASCVD). Nevertheless, IR is rarely measured in healthy individuals in a clinical setting because techniques for direct measurement of IR are labor intensive and expensive. Indirect methods for IR assessment have not been validated. While some patients with clear indications for evaluation of T2D risk may have IR assessed by their clinicians, many other patients will likely remain unaware of their elevated IR measure and the potentially increased risk of T2D and ASCVD.

A variety of clinical measures have the potential to assist in the identification of individuals with IR. Body mass index (BMI) is strongly associated with IR, but not all insulin resistant patients are obese. IR is also associated with lipid and lipoprotein abnormalities that comprise high triglyceride (TG) and low high-density lipoprotein cholesterol (HDL-C) concentrations and a preponderance of small dense low-density lipoprotein (LDL) particles. TG to HDL-C concentration ratio (TG/HDL-C) can be used to identify insulin resistant individuals. Lipoprotein size and LS concentrations have also been employed in the identification of persons with IR. In that context, an IR score based on nuclear magnetic resonance (NMR)-derived lipoprotein information was shown to have a strong association with multiple measures of IR. LS can also be measured by ion mobility. LS quantified by NMR and ion mobility are correlated, but not identical. Ion mobility-based methods are a direct measure of lipoprotein particle counts according to their size, while NMR is an algorithmically derived measurement.

The association of ion mobility-based LS (LS-IM) with a direct measure of IR has not been previously reported. Finding a strong association may provide additional information to patients and physicians about IR-driven risk of T2D and ASCVD. Therefore, we set out to describe the relationship between LS-IM and a direct measure of IR measured during the insulin suppression test and to determine the usefulness of LS to identify insulin resistant individuals.

### Research Design and Methods

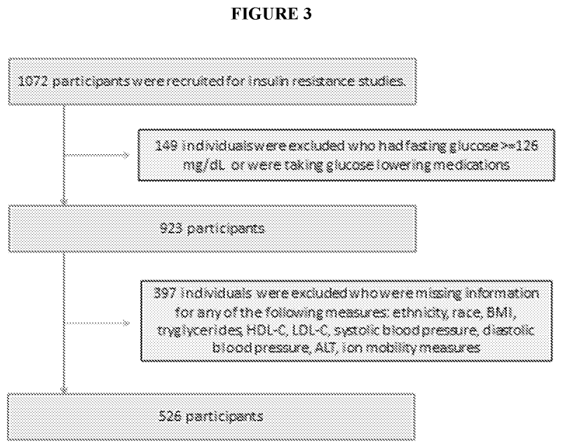

Study Population: This cross-sectional analysis includes 526 participants derived from 1072 apparently healthy individuals who had volunteered to participate in studies of IR between 1999 and 2011. Participants were recruited from the San Francisco Bay Area through advertisements in the local newspapers. The studies excluded pregnant women, individuals older than 79 or younger than 18 years, persons with history of cardiovascular disease, and patients with diabetes requiring insulin treatment. For this analysis, we excluded 149 participants who had fasting glucose ≥126 mg/dL and 397 participants with missing data for at least one of the following measures: race, ethnicity, body mass index (BMI), TG, HDL-C, LDL cholesterol, systolic blood pressure, diastolic blood pressure, alanine transaminase, or any of the ion mobility LS (FIG. 3).

The Institutional Review Board approved all studies, and all participants gave written informed consent.

### Clinical and Measurements

The study visits were conducted at Stanford Clinical and Translational Research Unit. Race and ethnicity were self-reported. Height and weight were measured without shoes and in light clothing; and BMI was calculated by dividing weight in kilograms by height in meters squared. Blood pressure was measured by an automatic blood pressure recorder after participants were seated quietly in a chair for 5 minutes with their feet on the floor and their arm supported at heart level. Three blood pressure measurements were obtained at 1-minute intervals using an appropriately sized cuff and were averaged.

### Insulin Suppression Test

The degree of IR was directly measured by the modified and validated version of the Insulin Suppression Test (IST), which quantifies the ability of a steady-state of physiological hyperinsulinemia to stimulate glucose uptake.

After an overnight fast, an intravenous catheter was placed in each arm. One arm was used for drawing blood samples and the other for giving a continuous infusion of octreotide acetate (0.27 μg/m²/min), insulin (32 mU/m²/min), and glucose (267 mg/m²/min) for 180 minutes. Blood was sampled every 30 minutes for 150 minutes and then every 10 minutes to measure steady-state plasma insulin (SSPI) and glucose (SSPG) concentrations.

During the IST, octreotide acetate inhibits endogenous insulin secretion and the infusion of insulin results in similar SSPI concentration (physiological hyperinsulinemia) among all individuals. The ability of physiological hyperinsulinemia to stimulate uptake of infused glucose is indicated by the SSPG concentration. The higher the SSPG concentration, the lower the insulin-stimulated glucose uptake, and the more insulin resistant a person. IR measured during the IST highly correlates with that measured during the euglycemic, hyperinsulinemic clamp test. Individuals in the top tertile of SSPG concentration were defined as being insulin resistant. This decision was based on the results of a prospective study where subjects in the tertile with the highest SSPG concentration developed more ASCVD than those in the tertile with the lowest SSPG concentration.

### Lipid and Lipoprotein Measurements

Lipid panel were assessed after overnight fasting measured at Stanford Health Care Clinical Laboratory and the Friedewald equation was used to calculate LDL cholesterol.

### Ion Mobility Measurements

LS levels were assessed by ion mobility at Quest Diagnostics Nichols Institute (San Juan Capistrano, CA). [Mora S, Caulfield M P, Wohlgemuth J, Chen Z, Superko H R, Rowland C M, Glynn R J, Ridker P M, Krauss R M: Atherogenic lipoprotein subfractions determined by ion mobility and first cardiovascular events after random allocation to high-intensity statin or placebo. 2015; 132:2220-2229; and Mora S, Caulfield M P, Wohlgemuth J, Chen Z, Superko H R, Rowland C M, Glynn R J, Ridker P M, Krauss R M. Atherogenic lipoprotein subfractions determined by ion mobility and first cardiovascular events after random allocation to high-intensity statin or placebo: the justification for the use of statins in prevention: an intervention trial evaluating rosuvastatin (JUPITER) Trial. Circulation 2015; 132:2220-9]. The LS and their definitions are provided in Table 3.

### Statistical Methods:

Pearson's correlation coefficient (r) was used as a measure of pairwise correlation. The associations of the TG/HDL-C and each LS-IM measure with SSPG concentration were assessed in separate linear regression models adjusting for age, sex, race, ethnicity and BMI. To incorporate multiple ion mobility variables and covariates in a single model, a backward stepwise regression model was performed using the Aikaike Information Criterion (AIC) as the metric to compare models. In the regression model, candidate variables were age, sex, race, ethnicity, BMI, TG/HDL-C and LS-IM measures and the dependent variable was SSPG concentration. Using the regression coefficients for the LS-IM measures in the final stepwise model, an ion mobility score (LS-IM score) was calculated. The score for each subject was a linear combination of the LS-IM variables from the stepwise model calculated as B1*Var1+B2*Var2+ . . . +Bp*Varp where B1 to Bp are the regression coefficients and Var1 to Varp are the subject specific values for p LS-IM variables in the final model. In a similar fashion, scores were calculated for other combinations of variables from the model: 1) LS-IM score+TG/HDL-C; 2) all non-ion mobility variables (sex, race, ethnicity, BMI and TG/HDL-C); and 3) all variables in the full model (sex, race, ethnicity, BMI, TG/HDL-C and LS-IM score). All continuous variables were standardized by transform to standard deviation (SD) units when included in the regression models. Tertiles of the LS-IM score, calculated using the ion mobility coefficients of the stepwise model, were plotted against SSPG concentration in tertiles of BMI for women and men.

Receiver operating characteristic (ROC) curves were plotted and the area under the curve (AUC) and 95% confidence intervals were calculated using Delong's method for each of the scores discussed above. Differences in AUC were assessed using Delong's method for paired ROC curves. [DeLong E R, DeLong D M, Clarke-Pearson D L. Comparing the areas under two or more correlated receiver operating characteristic curves: a nonparametric approach. Biometrics 1988; 44 (3): 837-45.] The positive predictive value (PPV) of identifying individuals in the top tertile of SSPG concentration was determined for each of the scores when considering the highest 5% of values for a score as a positive test. Wilson's method was used to calculate confidence intervals for the PPV. [Wilson E B. Probable Inference, the Law of Succession, and Statistical Inference. J Am Statistical Assoc 1927; 22:158.https://doi.org/10.1080/01621459.1927.10502953.209-212]. The Bonferroni method was used to determine significance levels adjusting for multiple comparisons. [Bland J M, Altman D G. Multiple significance tests: the Bonferroni method. BMJ 1995; 310:6973.https://doi.org/10.1136/bmj.310.6973.170 (Clinical research ed.) 170].

All analyses were performed using the R programming language. [Team RC: R: A Language and Environment for Statistical Computing. R Foundation for Statistical Computing. 2020]

### Results

The median age of study participants was 50 years and about two-thirds (65%) were women (Table 4). The majority of subjects were non-Hispanic (92%) and 70% were white. Nearly half (48%) of participants were obese (BMI≥30.0 kg/m²) and 38% were overweight (BMI 25.0 to 29.9 kg/m²).

It was found that there was a positive correlation between SSPG concentration and both BMI and TG/HDL-C(r=0.54 and 0.32 respectively) (Table 5).

Pairwise correlations (r) between BMI and the various LS-IM measures were less than 0.2 in absolute magnitude for all LS-IM measures and the only measure to reach statistical significance (p-value<0.0008) was HDL large which was negatively correlated with BMI (r=−0.17). Pairwise correlations between SSPG concentration and the individual LS-IM measures reaching statistical significance (p-value<0.0008) included: negative correlations of SSPG concentration with LDL peak particle size (r=−0.26), IDL small (r=−0.21), HDL large (r=−0.18), and LDL large a (r=−0.17); and positive correlations of SSPG concentration with LDL small (r=0.27), LDL very small a (r=0.23), LDL total (r=0.19), LDL medium (r=0.19) and LDL very small b (r=0.17). Stronger correlations were found between TG/HDL-C and the LS-IM measures: significant correlations (p-value<0.0008) were found for the following measures: negative correlations of TG/HDL-C with LDL peak particle size (r=−0.72), LDL Large a (r=−0.50), IDL small (r=−0.38), LDL large b (r=−0.37), HDL large (r=−0.33), HDL total (r=−0.18); and positive correlations of TG/HDL-C with LDL very small b (r=0.64), LDL very small a (r=0.62), LDL very small c (r=0.60), LDL Small (r=0.50), VLDL large (r=0.46), LDL very small d (r=0.38), VLDL medium (r=0.35), LDL total (r=0.32), and Non HDL total (r=0.23).

The majority of LS-IM measures were associated with a significant change in SSPG concentration as demonstrated by confidence intervals that did not span zero (Table 6). The largest changes per SD in LS-IM measures were for LDL peak particle size (SSPG concentration decreased by 16.7 mg/dL per 1 SD increase in peak particle size) and LDL Small (SSPG concentration increased by 15.8 mg/dL per 1 SD increase in LDL small particle number). Large effect sizes were also found for the non LS-IM measures of TG/HDL-C and BMI. Similarly, SSPG concentration increased by 17 mg/dL per 1SD increase in TG/HDL-C. In these same models, SSPG concentration increased by an average of 40 mg/dL per 1 SD increase in BMI.

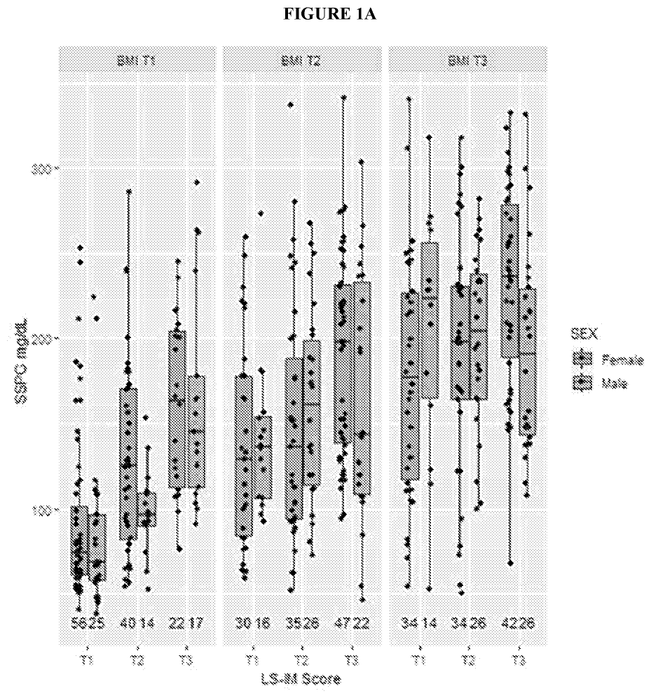

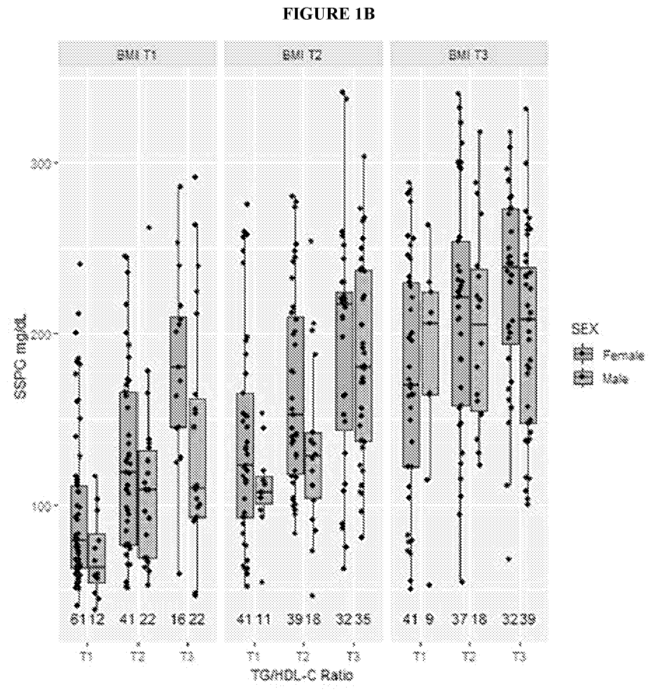

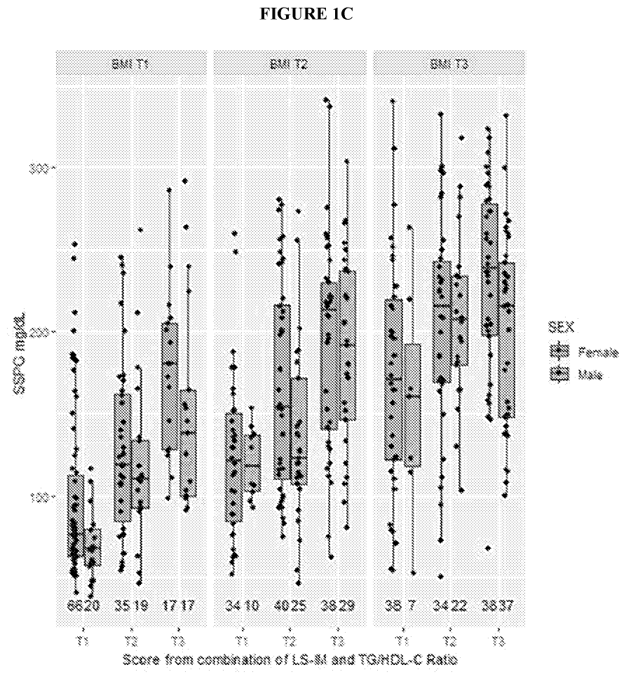

Eight of the LS-IM measures, as well as BMI, TG/HDL-C, sex, ethnicity, and race were the final variables remaining in a backwards stepwise regression analysis with SSPG concentration as a dependent variable (Table 1). Tertiles of the LS-IM score showed a strong positive relationship across all tertiles of BMI in women and in the lower two tertiles of BMI in men (FIG. 1A) and the relationship appears similar when tertiles of TG/HDL-C or tertiles of the score representing the combination of LS-IM+TG/HDL-C are plotted (FIGS. 1B and 1C).

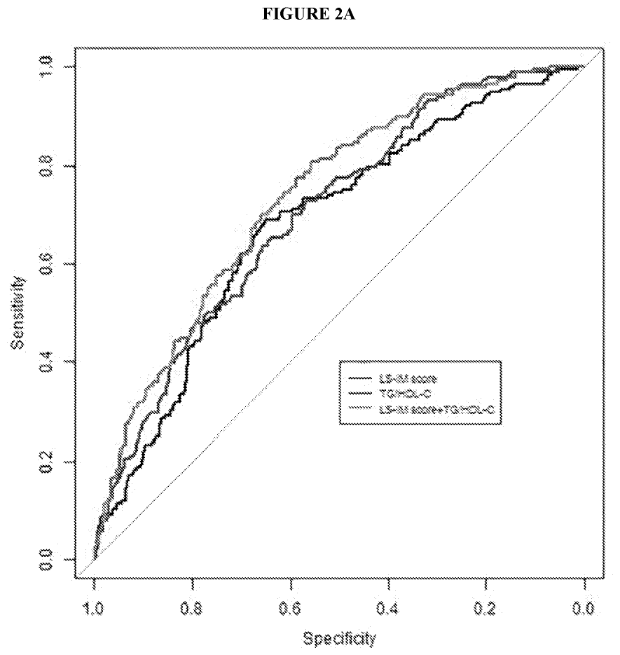

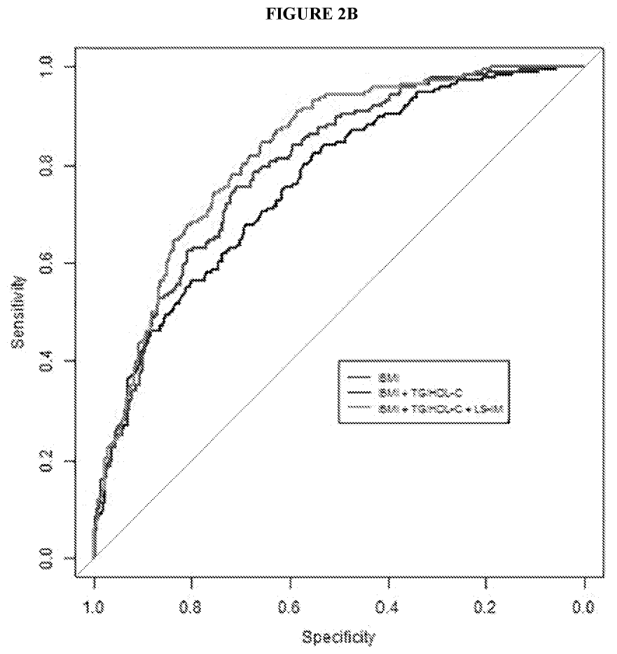

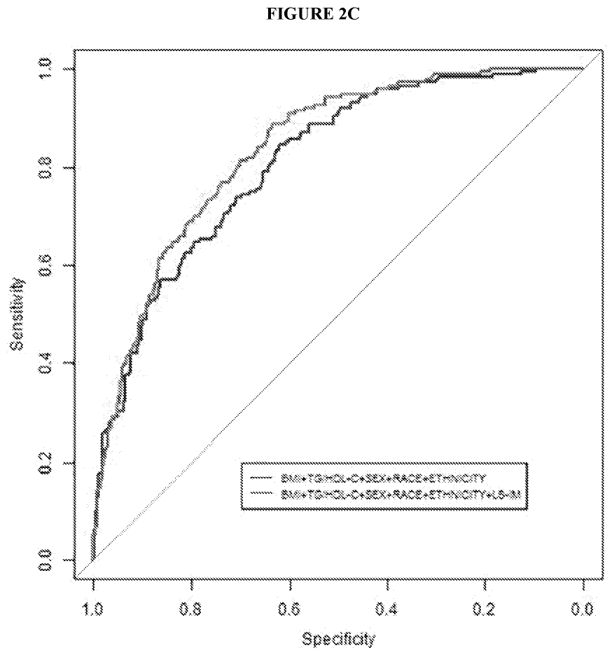

Receiver operating characteristic (ROC) curves for predicting IR (SSPG concentration in the top tertile) were plotted for the LS-IM score, TG/HDL-C, and LS-IM score+TG/HDL-C(FIG. 2A); BMI, BMI+TG/HDL-C and LS-IM score+BMI+TG/HDL-C(FIG. 2B); BMI+TG/HDL-C+Sex+Race+Ethnicity and LS-IM score+BMI+TG/HDL-C+Sex+Race+Ethnicity (FIG. 2C). The combination of LS-IM score+TG/HDL-C improved prediction of IR compared with TG/HDL-C alone (AUC=0.73 vs 0.70 respectively; p-value<0.0001) (Table 2). Similarly LS-IM score+BMI+TG/HDL-C improved prediction compared with BMI+TG/HDL-C alone (AUC=0.82 vs 0.79 respectively; p-value<0.0001) and LS-IM score also resulted in improved prediction when added to BMI+TG/HDL-C+Sex+Race+Ethnicity (AUC=0.84 vs 0.81 respectively; p-value<0.0001). In the subset of non-obese individuals, the additions of LS-IM score to TG/HDL-C, BMI+TG/HDL-C, and BMI+TG/HDL-C+Sex+Race+Ethnicity also resulted in improved prediction (p-value<0.0001) (Table 2).

The positive predictive values (PPV) were calculated for identifying subjects in the top tertile of SSPG concentration when considering the highest five percent of values for each of the same groups of variables. The PPVs ranged from 59% when considering TG/HDL-C or the IM score alone to 89% when considering the full model of LS-IM score+BMI+TG/HDL-C+Sex+Race+Ethnicity (Table 2).

### CONCLUSIONS

This study of LS and IR has three main findings. First, LS-IM had weak to modest correlations with a direct measure of IR (SSPG concentration). Second, prediction of IR by LS-IM was similar to that by TG/HDL-C. Third, prediction of IR improved when the LS-IM score was combined with TG/HDL-C and further improved by the addition of sex, race, ethnicity and BMI.

The role of IR in the pathogenesis of T2D and ASCVD and its association with metabolic abnormalities including elevated TG and low HDL-C concentrations was first formulated and presented by Reaven in 1988. Subsequent work by Reaven and colleagues and others demonstrated the association of IR with a myriad of metabolic abnormalities and various clinical syndromes including non-alcoholic fatty liver disease, obstructive sleep apnea, polycystic ovarian syndrome, and certain types of cancer that have deleterious health consequences. Thus, identification of IR in individuals is of clinical importance because it could prompt changes in behavior and clinical management to reduce risk associated with IR.

As methods for direct measurement of IR are unfeasible in a clinical setting, there is a need for robust and validated clinical measures of IR to identify individuals at risk for adverse consequences of IR. In this study, we aimed to fulfill this need by determining the usefulness of LS-IM measured by ion mobility to identify individuals with IR.

Dyslipidemia of IR is characterized by elevated TG and low HDL-C concentrations as well as by a preponderance of small dense LDL, postprandial lipemia, and increased concentration of partially oxidized LDL. Several lipid and LS abnormalities measured by NMR are also seen in persons with IR. Consistent with these previously reported findings, we show that several of the LS-IM measures were associated with IR (SSPG concentration). Specifically, we found that SSPG concentration was associated with increased number of large VLDL, medium to very small LDL and large HDL particles, and with smaller peak particle size and smaller number of small IDL, large LDL and large HDL particles. From a pathophysiological perspective, these associations are thought to arise in part from increased hepatic production and reduced clearance of VLDL from plasma as well as from increased hepatic lipase activity and subsequent hydrolysis of phospholipids in LDL and HDL particles leading to smaller and denser LDL particles and a decrease in large HDL particles and an increase in small HDL particles.

We also show that the incorporation of LS-IM improved prediction of IR as measured by SSPG concentration. Specifically, when predicting individuals in the top tertile of SSPG concentration, the AUC and PPV for the LS-IM score alone and TG/HDL-C alone were similar, but when used together they significantly improved the AUC and PPV (Table 2). Furthermore, the score obtained from the full stepwise model that included the LS-IM score with sex, race, ethnicity, BMI and TG/HDL-C had significantly improved the AUC and PPV compared to the score that excluded the LS-IM score and only included sex, race, ethnicity, BMI and TG/HDL-C. It is difficult to compare the LS-IM score described here with the previously described LP-IR score derived from NMR. The LP-IR score was based on HOMA-IR as a measure of IR while the current score was based on SSPG concentration, a direct measure of IR. In addition, the size ranges of the defined regions vary between the two scores. However, both scores demonstrate particles from a wide span of size ranges that independently contribute to the association with IR.

We also show that simpler measures of IR such as TG/HDL-C and BMI performed as well as the LS-IM score alone for prediction of IR. Specifically, we observed that TG/HDL-C ratio was similar to the LS-IM score and that BMI was similar to the combination of LS-IM score and TG/HDL-C for prediction of IR. We have previously shown that the TG/HDL-C can be used to identify insulin resistant individuals. TG/HDL-C ratio is a simple measure that can be used to identify individuals with increased cardiometabolic risk and would be preferable in clinical or research settings where the LS-IM measurements cannot be performed. Our results also demonstrate the potential utility of the LS-IM score to identify individuals who are most insulin resistant and have greater degrees of dyslipidemia in either obese or non-obese groups. . . . As shown in FIG. 1A, FIG. 1B, and FIG. 1C, within each tertile of BMI, individuals with higher scores (LS-IM, TG/HDL-C and LS-IM+TG/HDL-C) were generally more insulin resistant (higher SSPG concentration) than those with the lower LS-IM scores. As shown in Table 2, the AUCs for predicting IR among non-obese individuals were not diminished for LS-IM score, TG/HDL-C or their combination. This observation is consistent with our previous finding that, at a given BMI, insulin resistant individuals have higher TG and lower HDL-C concentrations than insulin sensitive persons.

Strengths of our study include the fact that we validated the usefulness of the LS-IM score for prediction of IR using a gold-standard measures of IR. In addition, we improved risk prediction using already available data from LS-IM clinical testing where those measurements are available (no additional cost). Limitations of our study include that the individuals in our study are not typical of the population undergoing LS-IM testing. The individuals studied were apparently healthy volunteers while those undergoing LS-IM testing are predominantly referred for testing by their clinicians for evaluation of risk of ASCVD. Future studies assessing the LS-IM score in patients undergoing LS-IM testing will be needed to evaluate the contribution of IR and the associated dyslipidemia towards ASCVD risk.

In conclusion, LS-IM measurements, in addition to TG/HDL-C and/or BMI, may improve prediction of IR. Among individuals who have undergone LS-IM testing, this information could be used to prioritize lifestyle interventions to improve IR and the associated risk of T2D and ASCVD. Targeted interventions including increased exercise and weight loss have been shown to be particularly helpful in improving IR and decreasing the progression of individuals to T2D. This information can also be used to identify individuals who may be candidates for further testing such as by other validated measures such as fasting insulin or the IR score and ultimately identify individuals who otherwise may be unaware of their IR and corresponding higher risk of T2D and ASCVD.

TABLE 1
Results of stepwise linear regression analysis

| Variable | Beta | 95% CI | P value |
|---|---|---|---|
| BMI | 33.5 | 28.4 to 38.6 | 8.71E−33 |
| Male (reference = Female) | −12.0 | −23.1 to −1 | 0.03 |
| Hispanic | 15.7 | −2.2 to 33.7 | 0.09 |
| (reference = Non-Hispanic) | | | |
| Race (reference = White) | | | |
| Native American | 5.3 | −39.8 to 50.4 | 0.82 |
| East Asian | 21.5 | 3.9 to 39.2 | 0.017 |
| Black | −6.0 | −24.9 to 12.8 | 0.53 |
| South Asian | 21.0 | 6.2 to 35.7 | 0.006 |
| TG/HDL-C | 17.1 | 9.8 to 24.4 | 5.86E−06 |
| VLDL Medium | −8.8 | −15.6 to −2.1 | 0.011 |
| IDL Small | −19.0 | −30 to −8.1 | 6.95E−04 |
| LDL Large a | 11.0 | −0.7 to 22.6 | 0.065 |
| LDL Medium | 10.2 | 4.5 to 15.9 | 4.49E−04 |
| LDL Very Small b | 14.8 | 5.1 to 24.6 | 0.003 |
| LDL Very Small c | −16.8 | −28.4 to −5.1 | 0.005 |
| LDL Very Small d | 8.6 | 0.4 to 16.8 | 0.041 |
| HDL Small | 7.9 | 2.2 to 13.6 | 0.007 |

Beta represents the change in SSPG concentration (mg/dL) per each one SD change (continuous variables) or from the reference category (categorical variables).
SD for continuous variables are: BMI = 5.4, TG/HDL-C = 2.45, VLDL Medium = 15.8, IDL Small = 50.3, LDL Large a = 92.6, LDL Medium = 8.1, LDL Very Small b = 62.6, LDL Very Small c = 34.6, LDL Very Small d = 19.5 and HDL Small = 3267 with all lipoprotein measures in nmol/L.
R-squared of model = 0.43
HDL: High-density lipoprotein
LDL: Low-density lipoprotein
IDL: Intermediate-density lipoprotein
VLDL: Very low-density lipoprotein

TABLE 2
Area under ROC curve (AUC) and positive predictive values (PPV) for predicting
individuals in the top tertile of SSPG concentration (>196 mg/dL)

| | | | | | R- |
|---|---|---|---|---|---|
| Strata | Model | AUC (95% CI) | P-value* | PPV† (95% CI) | squared‡ |
| All | LS-IM score | 0.68 (0.63 to 0.73) | | 0.59 (0.41 to 0.75) | 0.12 |
| | TG/HDL-C | 0.70 (0.65 to 0.75) | | 0.59 (0.41 to 0.75) | 0.10 |
| | LS-IM score + | 0.73 (0.69 to 0.78) | <0.0001^{a} | 0.70 (0.52 to 0.84) | 0.21 |
| | TG/HDL-C | | | | |
| | BMI | 0.76 (0.55 to 0.80) | | 0.74 (0.72 to 0.87) | 0.29 |
| | BMI + TG/HDL-C | 0.79 (0.76 to 0.83) | | 0.81 (0.63 to 91.8) | 0.35 |
| | LS-IM score + | 0.82 (0.79 to 0.86) | <0.0001^{a} | 0.81 (0.63 to 91.8) | 0.41 |
| | BMI + TG/HDL-C | | | | |
| | BMI + TG/HDL-C + | 0.81 (0.77 to 0.85) | | 0.85 (0.68 to 0.94) | 0.38 |
| | Sex + Race + Ethnicity | | | | |
| | LS-IM score + BMI + | 0.84 (0.80 to 0.87) | <0.0001^{a} | 0.89 (0.72 to 0.96) | 0.43 |
| | TG/HDL-C + Sex + | | | | |
| | Race + Ethnicity | | | | |
| BMI < 30 | LS-IM score | 0.77 (0.70 to 0.84) | | NA^{b} | 0.18 |
| | TG/HDL-C | 0.72 (0.65 to 0.79) | | NA^{b} | 0.07 |
| | LS-IM | 0.79 (0.72 to 0.85) | <0.0001^{a} | NA^{b} | 0.24 |
| | score + TG/HDL-C | | | | |
| | BMI | 0.71 (0.64 to 0.79) | | NA^{b} | 0.17 |
| | BMI + TG/HDL-C | 0.78 (0.71 to 0.85) | | NA^{b} | 0.21 |
| | LS-IM score + | 0.82 (0.76 to 0.88) | <0.0001^{a} | NA^{b} | 0.32 |
| | BMI + TG/HDL-C | | | | |
| | BMI + TG/HDL-C + | 0.78 (0.72 to 0.85) | | NA^{b} | 0.24 |
| | Sex + Race + Ethnicity | | | | |
| | LS-IM score + BMI + | 0.83 (0.77 to 0.89) | <0.0001^{a} | NA^{b} | 0.35 |
| | TG/HDL-C + Sex + | | | | |
| | Race + Ethnicity | 0.68 (0.63 to 0.73) | | | |

*P-value comparing AUC of current row with row immediately above. ^{a}indicates p-value is significant after adjusting for 6 statistical tests in table (<0.008)
†PPV when considering highest 5% of values as a positive test. ^{b}sample size of N = 13 among top 5% of non-obese subjects too small to obtain reliable PPV
‡R-squared of model with continuous SSPG concentration (mg/dL)
LS-IM Score = −8.8*VLDL Medium − 19*IDL Small + 11*LDL Large a + 10.2*LDL Medium + 14.8*LDL very small b − 16.8*LDL very small c + 8.6*LDL very small d + 7.9*HDL small
LS-IM Score + TG/HDL-C = LS-IM Score + 17.1*TG/HDL-C
BMI + TG/HDL-C = 33.5*BMI + 17.1*TG/HDL-C
BMI + TG/HDL-C + Sex + Race + Ethnicity = 33.5*BMI + 17.1*TG/HDL-C − 12*Male + 15.7*Hispanic + 5.3*Native American + 21.5*East Asian 6*Black + 21*South Asia
SSPG: Steady-state plasma glucose
TG/HDL-C: Triglyceride to high-density lipoprotein cholesterol ratio
LS-IM: Ion mobility based lipoprotein subfractionation
HDL: High-density lipoprotein
LDL: Low-density lipoprotein
IDL: Intermediate-density lipoprotein
VLDL: Very low-density lipoprotein

TABLE 3
Particle size range of ion mobility based lipoprotein subfractions

Particle Size Range (nm)

| Label | Minimum | Maximum |
|---|---|---|
| HDL Small | 7.65 | 10.5 |
| HDL Large | 10.5 | 14.5 |
| HDL Total | 7.65 | 14.5 |
| LDL Very Small d | 18 | 19 |
| LDL Very Small c | 19 | 19.9 |
| LDL Very Small b | 19.9 | 20.49 |
| LDL Very Small a | 20.49 | 20.82 |
| LDL Small | 20.82 | 21.41 |
| LDL Medium | 21.41 | 20.82 |
| LDL Medium − Small | 20.82 | 22 |
| LDL Large b | 22 | 22.46 |
| LDL Large a | 22.46 | 23.33 |
| LDL Total | 18 | 23.33 |
| LDL Peak Particle Size* | NA | NA |
| IDL Small | 23.33 | 25 |
| IDL Large | 25 | 29.6 |
| VLDL Small | 29.6 | 33.5 |
| VLDL Medium | 33.5 | 42.4 |
| VLDL Large | 42.4 | 52 |
| Non HDL Total | 18 | 52 |

*Particle diameter having the largest particle count observed in the LDL region

TABLE 4
Participant characteristics

| | All Participants | |
|---|---|---|
| Characteristic | (N = 526) | |
| Age (years) | 50.0 | [18.0, 71.0] |
| Sex | | |
| Female | 340 | (64.6) |
| Male | 186 | (35.4) |
| Race | | |
| White | 369 | (70.2) |
| South Asian | 69 | (13.1) |
| East Asian | 43 | (8.2) |
| Black | 39 | (7.4) |
| Native American | 6 | (1.1) |
| Ethnicity | | |
| Non-Hispanic | 484 | (92.0) |
| Hispanic | 42 | (8.0) |
| BMI (kg/m²) | 29.8 | [16.6, 60.8] |
| <25.0 | 75 | (14.3) |
| 25.0-29.9 | 199 | (37.8) |
| >=30.0 | 252 | (47.9) |
| SSPG (mg/dL) | 151.0 | [38.0, 341.0] |
| HDL-C (mg/dL) | 45 | [18, 108] |
| LDL-C (mg/dL) | 117 | [33, 240] |
| Triglycerides (mg/dL) | 108 | [15, 1217] |
| TG/HDL-C | 2.4 | [0.3, 28.3] |
| HDL Total (nmol/L) | 24088.5 | [14764.0, 59140.0] |
| HDL Small (nmol/L) | 19090.5 | [12135.0, 47704.0] |
| HDL Large (nmol/L) | 4849.0 | [2552.0, 11436.0] |
| Non HDL Total (nmol/L) | 1569.5 | [681.0, 3002.0] |
| LDL Total (nmol/L) | 1161.5 | [562.0, 2217.0] |
| LDL Peak Particle Size (Angstrom) | 218.7 | [199.0, 231.2] |
| LDL Very Small d (nmol/L) | 62.0 | [27.0, 181.0] |
| LDL Very Small c (nmol/L) | 74.0 | [27.0, 357.0] |
| LDL Very Small b (nmol/L) | 74.0 | [27.0, 480.0] |
| LDL Very Small a (nmol/L) | 68.5 | [20.0, 315.0] |
| LDL Medium + Small (nmol/L) | 429.0 | [160.0, 1100.0] |
| LDL Small (nmol/L) | 180.0 | [64.0, 597.0] |
| LDL Medium (nmol/L) | 236.0 | [93.0, 617.0] |
| LDL Large b (nmol/L) | 163.5 | [49.0, 367.0] |
| LDL Large a (nmol/L) | 222.5 | [70.0, 538.0] |
| IDL Small (nmol/L) | 146.0 | [49.0, 339.0] |
| IDL Large (nmol/L) | 140.0 | [38.0, 296.0] |
| VLDL Small (nmol/L) | 56.0 | [12.0, 132.0] |
| VLDL Medium (nmol/L) | 42.0 | [5.0, 99.0] |
| VLDL Large (nmol/L) | 11.0 | [1.0, 34.0] |

Values are N (%) or median [range]

TABLE 5
Pairwise Pearson correlation coefficients of SSPG,
BMI and ion mobility based lipoprotein subfractions

| Variable | SSPG | BMI | TG/HDL-C |
|---|---|---|---|
| SSPG | 1.00^{a} | 0.54^{a} | 0.32^{a} |
| BMI | 0.54^{a} | 1.00^{a} | 0.16^{a} |
| TG/HDL-C | 0.32^{a} | 0.16^{a} | 1.00^{a} |
| LDL Peak Particle Size | −0.26^{a} | −0.11 | −0.72^{a} |
| HDL Total | −0.01^{a} | −0.05 | −0.18^{a} |
| LDL Total | 0.19^{a} | 0.08 | 0.32^{a} |
| Non HDL Total | 0.11^{b} | 0.04 | 0.23^{a} |
| HDL Small | 0.07 | 0.01 | −0.09 |
| HDL Large | −0.18^{a} | −0.17^{a} | −0.33^{a} |
| LDL Very Small d | 0.08 | −0.08 | 0.38^{a} |
| LDL Very Small c | 0.10 | −0.05 | 0.60^{a} |
| LDL Very Small b | 0.17^{a} | 0.04 | 0.64^{a} |
| LDL Very Small a | 0.23^{a} | 0.09 | 0.62^{a} |
| LDL Small | 0.27^{a} | 0.13^{b} | 0.50^{a} |
| LDL Medium | 0.19^{a} | 0.13^{b} | 0.05 |
| LDL Medium − Small | 0.26^{a} | 0.14^{b} | 0.33^{a} |
| LDL Large b | −0.02 | 0.03 | −0.37^{a} |
| LDL Large a | −0.17^{a} | −0.05 | −0.50^{a} |
| IDL Small | −0.21^{a} | −0.07 | −0.38^{a} |
| IDL Large | 0.00 | −0.02 | 0.09 |
| VLDL Small | −0.13^{b} | −0.13^{b} | 0.01 |
| VLDL Medium | −0.01 | −0.07 | 0.35^{a} |
| VLDL Large | 0.07 | −0.05 | 0.46^{a} |

^{a}p-value < 0.0008;
^{b}p-value < 0.01;

TABLE 6
Linear regression model results showing relationship
of triglyceride/HDL-C and individual ion mobility
based lipoprotein subfractions with steady state
plasma glucose concentration (SSPG)

| Variable | Beta | 95% CI | P value |
|---|---|---|---|
| Triglyceride/HDL-C | 17.0 | 11.9 to 22.2 | 2.43E−10 |
| Non HDL Total | 6.4 | 1.2 to 11.6 | 0.017 |
| VLDL Large | 6.2 | 0.9 to 11.5 | 0.022 |
| VLDL Medium | 2.0 | −3.2 to 7.3 | 0.45 |
| VLDL Small | −4.2 | −9.4 to 1 | 0.11 |
| IDL Large | 0.7 | −4.4 to 5.9 | 0.78 |
| IDL Small | −12.8 | −18.1 to −7.5 | 2.46E−06 |
| LDL Peak Particle | −16.7 | −22 to −11.3 | 1.95E−09 |
| Size | | | |
| LDL Total | 10.5 | 5.3 to 15.7 | 8.40E−05 |
| LDL Large a | −11.1 | −16.4 to −5.9 | 4.24E−05 |
| LDL Large b | −3.3 | −8.5 to 1.9 | 0.21 |
| LDL Medium − Small | 13.7 | 8.6 to 18.9 | 2.62E−07 |
| LDL Medium | 8.4 | 3.3 to 13.5 | 0.0014 |
| LDL Small | 15.8 | 10.6 to 21 | 5.39E−09 |
| LDL Very Small a | 14.7 | 9.4 to 20 | 8.17E−08 |
| LDL Very Small b | 11.8 | 6.5 to 17.1 | 1.59E−05 |
| LDL Very Small c | 8.8 | 3.5 to 14 | 0.0012 |
| LDL Very Small d | 7.9 | 2.8 to 13 | 0.0026 |
| HDL Total | 0.4 | −4.9 to 5.8 | 0.88 |
| HDL Large | −8.3 | −13.8 to −2.7 | 0.0038 |
| HDL Small | 3.6 | −1.6 to 8.9 | 0.18 |

Beta: Change in SSPG concentration per each 1 SD increase of variable estimated in separate models adjusted for age, sex, ethnicity, race and body mass index

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

The methods illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof. It is recognized that various modifications are possible within the scope of the invention claimed. Thus, it should be understood that although the present invention has been specifically disclosed by preferred embodiments and optional features, modification and variation of the invention embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the methods. This includes the generic description of the methods with a proviso or negative limitation removing any subject matter from the genus, regardless of whether or not the excised material is specifically recited herein.

Other embodiments are within the following claims. In addition, where features or aspects of the methods are described in terms of Markush groups, those skilled in the art will recognize that the invention is also thereby described in terms of any individual member or subgroup of members of the Markush group.

1. A method for diagnosing or prognosing insulin resistance in a patient in need thereof, the method comprising determining the amount of lipoprotein subfraction in a sample by ion mobility.

2. The method of claim 1, wherein the method further comprises measuring triglyceride (TG) levels.

3. The method of claim 1, wherein the method further comprises measuring high density lipoprotein cholesterol (HDL-C) levels.

4. The method of claim 1, wherein the method further comprises measuring body mass index (BMI) in combination with sex, race, and ethnicity.

5. The method of claim 1, wherein the method further comprises measuring triglyceride (TG) levels and high density lipoprotein cholesterol (HDL-C) levels.

6. The method of claim 1, wherein the method further comprises measuring triglyceride (TG) levels, high density lipoprotein cholesterol (HDL-C) levels, and body mass index (BMI) in combination with sex, race, and ethnicity.

7. The method of claim 1, wherein the method provides an insulin resistance score.

8. The method of claim 1, wherein the method provides a probability of developing insulin resistance.

9. The method of claim 1, wherein said sample comprises a plasma or serum sample.

10. The method of claim 1, wherein determining the amount of lipoprotein subfraction comprises determining the amount of one or more or all of VLDL (very low-density lipoprotein), IDL (intermediate-density lipoprotein), LDL (low-density lipoprotein), and HDL (high-density lipoprotein).

11. The method of claim 1, wherein determining the amount of lipoprotein subfraction comprises determining the amount of one or more or all of VLDL (very low-density lipoprotein) Medium, IDL (intermediate-density lipoprotein) Small, LDL (low-density lipoprotein) Large a, LDL (low-density lipoprotein) Medium, LDL (low-density lipoprotein) Very Small b, LDL (low-density lipoprotein) Very Small c, LDL (low-density lipoprotein) Very Small d, and HDL (high-density lipoprotein) Small.

12. A method for determining the amount of lipoprotein subfraction in a sample, the method comprising determining the amount of the lipoprotein subfraction in the sample by ion mobility.

13. The method of claim 12, wherein the method further comprises measuring triglyceride (TG) levels.

14. The method of claim 12, wherein the method further comprises measuring high density lipoprotein cholesterol (HDL-C) levels.

15. The method of claim 12, wherein the method further comprises measuring body mass index (BMI) in combination with sex, race, and ethnicity.

16. The method of claim 12, wherein the method further comprises measuring triglyceride (TG) levels and high density lipoprotein cholesterol (HDL-C) levels.

17. The method of claim 12, wherein the method further comprises measuring triglyceride (TG) levels, high density lipoprotein cholesterol (HDL-C) levels, and body mass index (BMI) in combination with sex, race, and ethnicity.

18. The method of claim 12, wherein the method provides an insulin resistance score.

19. The method of claim 12, wherein the method provides a probability of developing insulin resistance.

20. The method of claim 12, wherein said sample comprises a plasma or serum sample.

21. The method of claim 12, wherein determining the amount of lipoprotein subfraction comprises determining the amount of one or more or all of VLDL (very low-density lipoprotein), IDL (intermediate-density lipoprotein), LDL (low-density lipoprotein), and HDL (high-density lipoprotein).

22. The method of claim 12, wherein determining the amount of lipoprotein subfraction comprises determining the amount of one or more or all of VLDL (very low-density lipoprotein) Medium, IDL (intermediate-density lipoprotein) Small, LDL (low-density lipoprotein) Large a, LDL (low-density lipoprotein) Medium, LDL (low-density lipoprotein) Very Small b, LDL (low-density lipoprotein) Very Small c, LDL (low-density lipoprotein) Very Small d, and HDL (high-density lipoprotein) Small.
